(19) United States
(12) Patent Application Publication
(10) Pub. No.: US 2008/0261844 A1
(43) Pub. Date: Oct. 23, 2008

(54) Multicolor Cosmetics

(75) Inventors: Stephan Ruppert, Hamburg (DE); Michaela Kohut, Hamburg (DE)

Correspondence Address: GREENBLUM & BERNSTEIN, P.L.C., 1950 ROLAND CLARKE PLACE, RESTON, VA, 20191, US

(73) Assignee: BEIERSDORF AG, Hamburg (DE)

(21) Appl. No.: 11/574,616

(22) PCT Filed: Sep. 3, 2004
(86) PCT No.: PCT/EP2004/009847

§ 371 Date: Aug. 7, 2007

(51) Int. Cl. A61K 8/02; A61Q 19/10

(52) U.S. Cl. 510/158

(57) ABSTRACT

Multicolored cosmetic preparation containing
a) one or more pigment dyes,
b) one or more hydrocolloids,
c) water,
   optionally along with additional cosmetic or dermatological active agents, adjuvants and excipients,
   with a yield point of 0.5 Pa to 40 Pa and a tan δ of 0.02 to 0.8

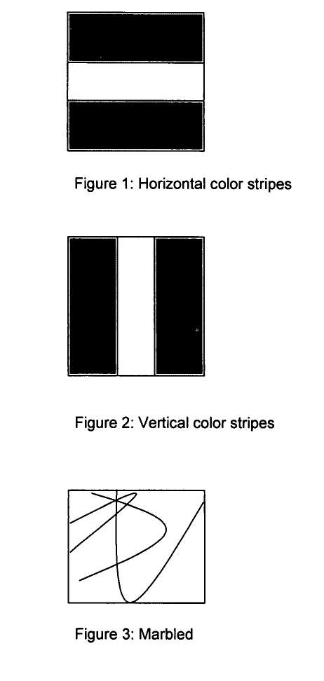

The present invention concerns multicolored cosmetic preparations.

The desire to appear beautiful and attractive is ingrained in humans by nature. Even if the ideal of beauty has changed over time, the attainment of a flawless exterior has always been the goal of humans, and an important part of a beautiful and attractive exterior is the condition and appearance of the skin and skin adnexa.

So that the skin and skin adnexa, which primarily include the hair and nails, can fulfill their biological functions to the full extent, they need regular cleansing and care. Cleansing serves to remove dirt, sweat and the residue of dead cells, which form an ideal growth environment for pathogens and parasites of all types. Cosmetic cleansing products are usually offered in the form of gels, lotions and solids (bars of soap, cleansing syndets). Skin care products, usually creams, salves or lotions, usually serve to moisturize and relubricate the skin. Frequently, substances are added which are supposed to regenerate the skin and, for example, prevent and reduce its premature aging (e.g. the appearance of crow's feet and wrinkles). UV light filters are added to many cosmetic and dermatological products to protect from the damaging UV radiation of sunlight.

Cosmetic formulations often represent externally uniform, homogenous preparations. But these are increasingly perceived by users as monotonous and boring. To make the preparations more attractive to users, color and effect materials can be added to them.

Cosmetic gels, for example, are usually transparent or translucent preparations. Cosmetic emulsions (e.g. shower emulsions containing surfactants and oils) are usually white. To make these products more visually attractive to the user, they can be dyed.

Products that are usually offered in transparent packaging can also be given interesting visual effects by incorporating color particles, gas bubbles, substance capsules, glittering substances and other larger objects (generally called effect materials). Such formulations are especially attractive for children and adolescents, who find the customary colorless cleansers unattractive and boring and frequently regard bathing and washing as superfluous and burdensome.

Multicolored cosmetic preparations, especially liquid or viscous cleansing preparations, are unknown up to now in the state of the art, for it is problematic, since color effects are mostly unstable over a longer period of time. Storage, transport and temperature stability especially leave much to be desired with regard to multicolored preparations.

If soluble dyes are used in a multicolored preparation, or if only a part of the formulation is to be dyed, diffusion causes a mixing of the individual color zones of the preparation. The sharp color boundaries dissolve to form a flowing color continuum, which many users interpret to mean that the preparation is unusable and “spoiled”. As a rule, soluble dyes in the preparation are also more easily absorbed by the skin than color pigments, which is usually undesirable from a toxicological or allergological point of view.

The use of pigment dyes in multicolored preparations according to the state of the art is also flawed by a series of disadvantages. Usually, these dyes can only be used in solid or highly viscous preparations (e.g. bars of soap, cleansing syndets, toothpaste) since the pigments settle over time in less viscous preparations. The sinking of the particles to the bottom of the packaging is also accelerated by vibrations (e.g. during transport), so that such products lack storage and transport stability. The manufacturing of stable, multicolored shower gels, hair shampoos, hair conditioners or styling gels was not possible according to the state of the art.

It was therefore surprising and unforeseeable for the person skilled in the art that multicolored cosmetic preparations containing

a) one or more pigment dyes,
b) one or more gel formers,
c) water,
and optionally additional cosmetic or dermatological agents, adjuvants and excipients, with a yield point of from 0.5 Pa to 40 Pa and a tan δ of from 0.02 to 0.8, remedy the deficiencies of the state of the art.

The preparations according to the invention are characterized by an unusually high stability. Storage and transport stability in particular, but also temperature stability, are significantly increased. Even thin layers of color in the preparation, as present in partially stirred, marbled preparations or with striations, remain stable in the formulation over a long period of time. Illustrations 1, 2 and 3 show some embodiments of the invention according to the invention.

Here, the critical shear stress of the flow curve is regarded as the yield point according to the invention. It can be determined according to the invention as follows:

The flow curve is measured on a controlled shear stress rheometer at 25° C.±1° C. with 25 mm plate/plate geometry with a gap between 0.8 mm and 1.2 mm, which is filled in a manner that preserves the structure. A suitable constant shear stress time ramp is predetermined; a corresponding structural relaxation time is maintained before the test and the critical shear stress is indicated as the maximum of the flow curve.

Filling in a manner that preserves the structure is understood as filling that takes place as follows: The product is carefully taken up in a scoop and put into the measuring device. The product should not be sheared when doing this to avoid destroying or influencing the structures.

A tan δ according to the invention is understood as the quotient of the loss modulus and the storage modulus.

The tan δ is determined as follows:

The loss modulus and storage modulus are measured by a dynamic frequency test on a controlled shear stress rheometer at 40° C.±1° C. with 25 mm plate/plate geometry and a gap between 0.8 mm and 1.2 mm, which is filled in a manner that preserves the structure. The frequency test is carried out according to the state of the art, with a suitable structural relaxation time prior to the test, and the tan δ is given in the frequency range between 0.05 rad/s and 3.0 rad/s, preferably between 0.08 rad/s and 1.0 rad/s.

According to the invention, a yield point of 0.5 to 20 Pa is preferred, with a yield point of 1 to 6 Pa being particularly preferred.

It is preferred, according to the invention, for tan δ to lie between 0.05 and 0.6, with the range from 0.1 to 0.5 being particularly preferred.

To achieve this, the yield point and tan δ according to the invention are adjusted by varying the percentage of gel former in the formulation.

It is advantageous if the multicolored cosmetic preparation according to the invention contains one or more pigment dyes in a quantity of from 0.001 to 2 wt. %, preferably from 0.001 to 1 wt. % and particularly preferred from 0.001 to 0.5 wt. % relative to the total weight of the preparation.

Of course, these quantities also apply for partial portions of the preparation.

Partial portions are understood to be individual portions of the preparation according to the invention into which the uncolored (but not necessarily colorless) raw preparation is divided, and which are dyed with different pigment dyes. Here, it may prove advantageous according to the invention if one of the partial preparations remains undyed. Thus, it can be advantageous according to the invention if the partial portions are identical in their composition, except for the type and content of pigment dyes.

This is not obligatory, however, since the partial preparations can also have different compositions, which is preferred according to the invention.

All compounds listed in the corresponding definitive list of the Cosmetics Ordinance and the EC List of cosmetic dyes can be used as pigment dyes which are advantageous according to the invention. In most cases, they are identical with the dyes approved for use in food. Along with color pigments, the preparations according to the invention can also contain additional dyes. Advantageous color pigments are, for example, titanium dioxide, mica, iron oxides (e.g. Fe₂O₃, Fe₃O₄, FeO(OH)) and/or tin oxide. Advantageous dyes are, for example, carmine, Berlin blue, chromium oxide green, ultramarine blue and/or manganese violet. It is especially advantageous to choose the dyes and/or color pigments from the following list. The Color Index Numbers (CIN) are taken from the Rowe Colour Index, 3^{rd} Edition, Society of Dyers and Colourists, Bradford, England, 1971.

| Chemical or other name | CIN | Color |
|---|---|---|
| Pigment Green | 10006 | green |
| Acid Green 1 | 10020 | green |
| 2,4-Dinitrohydroxynaphthalene-7-sulfonic acid | 10316 | yellow |
| Pigment Yellow 1 | 11680 | yellow |
| Pigment Yellow 3 | 11710 | yellow |
| Pigment Orange 1 | 11725 | orange |
| 2,4-Dihydroxyazobenzene | 11920 | orange |
| Solvent Red 3 | 12010 | red |
| 1-(2′-Chloro-4′-nitro-1′-phenylazo)-2-hydroxynaphthalene | 12085 | red |
| Pigment Red 3 | 12120 | red |
| Ceres red; Sudan red; Fat Red G | 12150 | red |
| Pigment Red 112 | 12370 | red |
| Pigment Red 7 | 12420 | red |
| Pigment Brown 1 | 12480 | brown |
| 4-(2′-methoxy-5′-sulfodiethylamido-1′-phenylazo)-3-hydroxy- | 12490 | red |
| 5″-Chloro-2″,4″-dimethoxy-2-naphthanilide | | |
| Disperse Yellow 16 | 12700 | yellow |
| 1-(4-Sulfo-1-phenylazo)-4-aminobenzene-5-sulfonic acid | 13015 | yellow |
| 2,4-Dihydroxyazobenzene-4′-sulfonic acid | 14270 | orange |
| 2-(2,4-Dimethylphenylazo-5-sulfo)-1-hydroxynaphthalene-4- | 14700 | red |
| sulfonic acid | | |
| 2-(4-Sulfo-1-naphthylazo)-1-naphthol-4-sulfonic acid | 14720 | red |
| 2-(6-Sulfo-2,4-xylylazo)-1-naphthol-5-sulfonic acid | 14815 | red |
| 1-(4′-Sulfophenylazo)-2-hydroxynaphthalene | 15510 | orange |
| 1-(2-Sulfo-4-chloro-5-carboxy-1-phenylazo)-2- | 15525 | red |
| hydroxynaphthalene | | |
| 1-(3-Methyl-phenylazo-4-sulfo)-2-hydroxynaphthalene | 15580 | red |
| 1-(4′,(8′)-Sulfonaphthylazo)-2-hydroxynaphthalene | 15620 | red |
| 2-Hydroxy-1,2′-azonaphthalene-1′-sulfonic acid | 15630 | red |
| 3-Hydroxy-4-phenylazo-2-naphthylcarboxylic acid | 15800 | red |
| 1-(2-Sulfo-4-methyl-1-phenylazo)-2-naphthylcarboxylic acid | 15850 | red |
| 1-(2-Sulfo-4-methyl-5-chloro-1-phenylazo)-2- | 15865 | red |
| hydroxynaphthalene-3-carboxylic acid | | |
| 1-(2-Sulfo-1-naphthylazo)-2-hydroxynaphthalene-3-carboxylic acid | 15880 | red |
| 1-(3-Sulfo-1-phenylazo)-2-naphthol-6-sulfonic acid | 15980 | orange |
| 1-(4-Sulfo-1-phenylazo)-2-naphthol-6-sulfonic acid | 15985 | yellow |
| Allura Red | 16035 | red |
| 1-(4-Sulfo-1-naphthylazo)-2-naphthol-3,6-disulfonic acid | 16185 | red |
| Acid Orange 10 | 16230 | orange |
| 1-(4-Sulfo-1-naphthylazo)-2-naphthol-6,8-disulfonic acid | 16255 | red |
| 1-(4-Sulfo-1-naphthylazo)-2-naphthol-3,6,8-trisulfonic acid | 16290 | red |
| 8-Amino-2-phenylazo-1-naphthol-3,6-disulfonic acid | 17200 | red |
| Acid Red 1 | 18050 | red |
| Acid Red 155 | 18130 | red |
| Acid Yellow 121 | 18690 | yellow |
| Acid Red 180 | 18736 | red |
| Acid Yellow 11 | 18820 | yellow |
| Acid Yellow 17 | 18965 | yellow |
| 4-(4-Sulfo-1-phenylazo)-1-(4-sulfophenyl)-5-hydroxy- | 19140 | yellow |
| pyrazolone-3-carboxylic acid | | |
| Pigment Yellow 16 | 20040 | yellow |
| 2,6-(4′-Sulfo-2″,4″-dimethyl)-bisphenylazo)1,3- | 20170 | orange |
| dihydroxybenzene | | |
| Acid Black 1 | 20470 | black |
| Pigment Yellow 13 | 21100 | yellow |
| Pigment Yellow 83 | 21108 | yellow |
| Solvent Yellow | 21230 | yellow |
| Acid Red 163 | 24790 | red |
| Acid Red 73 | 27290 | red |
| 2-[4′-(4″-Sulfo-1″-phenylazo)-7′-sulfo-1′-naphthylazo]-1- | 27755 | black |
| hydroxy-7-aminonaphthalene-3,6-disulfonic acid | | |
| 4′-[(4″-Sulfo-1″-phenylazo)-7′-sulfo-1′-naphthylazo]-1-hydroxy- | 28440 | black |
| 8-acetylaminonaphthalene-3,5-disulfonic acid | | |
| Direct Orange 34, 39, 44, 46, 60 | 40215 | orange |
| Food Yellow | 40800 | orange |
| trans-β-apo-8′-Carotene aldehyde (C₃₀) | 40820 | orange |
| Ethyl ester of trans-apo-8′-carotenic acid (C₃₀) | 40825 | orange |
| Canthaxanthin | 40850 | orange |
| Acid Blue 1 | 42045 | blue |
| 2,4-Disulfo-5-hydroxy-4′-4″-bis-(diethylamino)triphenylcarbinol | 42051 | blue |
| 4-[(-4-N-Ethyl-p-sulfobenzylamino)-phenyl-(4-hydroxy-2- | 42053 | green |
| sulfophenyl)-(methylene)-1-(N-ethylN-p-sulfobenzyl)-2,5- | | |
| cyclohexadienimine] | | |
| Acid Blue 7 | 42080 | blue |
| (N-Ethyl-p-sulfobenzyl-amino)-phenyl-(2-sulfophenyl)- | 42090 | blue |
| methylene-(N-ethyl-N-p-sulfo-benzyl)Δ^{2,5}-cyclohexadienimine | | |
| Acid Green 9 | 42100 | green |
| Diethyl-disulfobenzyl-di-4-amino-2-chloro-di-2- | 42170 | green |
| methylfuchsonimmonium | | |
| Basic Violet 14 | 42510 | violet |
| Basic Violet 2 | 42520 | violet |
| 2′-Methyl-4′-(N-ethyl-N-m-sulfobenzyl)-amino-4″-(N-diethyl)- | 42735 | blue |
| amino-2-methyl-N-ethylN-m-sulfobenzyl-fuchsonimmonium | | |
| 4′-(N-Dimethyl)-amino-4″-(N-phenyl)-aminonaphtho-N- | 44045 | blue |
| dimethylfuchsonimmonium | | |
| 2-Hydroxy-3,6-disulfo-4,4′-bis-dimethylamino- | 44090 | green |
| naphthofuchsonimmonium | | |
| Acid Red 52 | 45100 | red |
| 3-(2′-Methylphenylamino)-6-(2′-methyl-4′-sulfophenylamino)- | 45190 | violet |
| 9-(2″-carboxyphenyl)-xanthenium salt | | |
| Acid Red 50 | 45220 | red |
| Phenyl-2-oxyfluorone-2-carboxylic acid | 45350 | yellow |
| 4,5-Dibromofluorescein | 45370 | orange |
| 2,4,5,7-Tetrabromofluorescein | 45380 | red |
| Solvent Dye | 45396 | orange |
| Acid Red 98 | 45405 | red |
| 3′,4′,5′,6′-Tetrachloro-2,4,5,7-tetrabromofluorescein | 45410 | red |
| 4,5-Diiodofluorescein | 45425 | red |
| 2,4,5,7-Tetraiodofluorescein | 45430 | red |
| Chinophthalone | 47000 | yellow |
| Chinophthalone disulfonic acid | 47005 | yellow |

It can also be advantageous to choose one or more substances from the following group as a dye: 2,4-dihydroxyazobenzene, 1-(2′-chloro-4′-nitro-1′-phenylazo)-2-hydroxynaph-thalene, Ceres red, 2-(4-sulfo-1-naphthylazo)-1-naphthol-4-sulfonic acid, calcium salt of 2-hydroxy-1,2′-azonaphthalene-1′-sulfonic acid, calcium and barium salts of 1-(2-sulfo-4-methyl-1-phenylazo)-2-naphthylcarboxylic acid, calcium salt of 1-(2-sulfo-1-naphthylazo)-2-hydroxynaphthalene-3-carboxylic acid, aluminum salt of 1-(4-sulfo-1-phenylazo)-2-naphthol-6-sulfonic acid, aluminum salt of 1-(4-sulfo-1-naphthylazo)-2-naphthol-3,6-disulfonic acid, 1-(4-sulfo-1-naphthylazo)-2-naphthol-6,8-disulfonic acid, aluminum salt of 8-amino-2-phenylazo-1-naphthol-3,6-disulfonic acid, aluminum salt of 4-(4-sulfo-1-phenylazo)-1-(4-sulfophenyl)-5-hydroxypyrazolone-3-carboxylic acid, 4′-[(4″-sulfo-1″-phenylazo)-7′-sulfo-1′-naphthylazo]-1-hydroxy-8-acetylaminonaphthalene-3,5-disulfonic acid, aluminum and zirconium salts of 4,5-dibromofluorescein, aluminum and zirconium salts of 2,4,5,7-tetrabromofluorescein, 3′,4′,5′,6′-tetrachloro-2,4,5,7-tetrabromofluorescein and its aluminum salt, aluminum salt of 2,4,5,7-tetraiodofluorescein, aluminum salt of chinophthalone disulfonic acid, aluminum salt of indigo disulfonic acid, 4,4′-dimethyl-6,6′-dichlorothioindigo, complex salt (Na, Al, Ca) of carminic acid, red and black iron oxide (CIN: 77491 (red) and 77499 (black)), iron oxide hydrate (CIN: 77492), manganese ammonium diphosphate (CIN 77745), ultramarine (CIN 77007) and titanium dioxide.

Titanium dioxides according to the invention, which can be present in both the crystal modifications rutile and anatase, are advantageously surface-treated (coated) in the sense of the present invention, thus forming or maintaining, for example, a hydrophilic, amphiphilic or hydrophobic character. This surface treatment can consist of the pigments being provided with a thin inorganic and/or organic hydrophilic and/or hydrophobic layer according to processes already known. The different surface coatings can also contain water in the sense of the present invention.

Inorganic surface coatings in the sense of the present invention can consist of aluminum oxide (Al₂O₃), aluminum hydroxide Al(OH)₃, or aluminum oxide hydrate (also: alumina, CAS No.: 1333-84-2), sodium hexametaphosphate (NaPO₃)₆, sodium metaphosphate (NaPO₃)ₙ, silicon dioxide (SiO₂) (also: silica, CAS No.: 7631-86-9), zirconium oxide (ZrO₂) or iron oxide (Fe₂O₃). These inorganic surface coatings can occur in combination and/or in combination with organic coating materials.

For this, oxides, oxide hydrates or phosphates, for example, of the elements Al, Si, Zr in thick coats are precipitated onto the pigment surface.

The inorganic after-treatment is generally carried out in an aqueous suspension of the pigment by adding soluble after-treatment chemicals such as aluminum sulfate, and then precipitating the hydroxide, which is scarcely soluble in the neutral range, by targeted adjustment of the pH with sodium hydroxide.

Following the inorganic after-treatment, the coated pigments are separated from the suspension by filtration and carefully washed to remove the dissolved salts, and then the isolated pigments are dried.

Especially preferred in the sense of this invention are titanium dioxides to which aluminum hydroxide has been applied on the surface, such as the titanium dioxide types C47-051 and C47-5175 that can be obtained from Sun Chemical. Additional preferred pigments are titanium dioxides coated with aluminum and/or silicon oxides, such as the titanium from Krosnos: Kronos 1071 and 1075, or that from Kingfisher: A310.03 Tudor Aspen.

Organic surface coatings in the sense of this invention can consist of plant or animal aluminum stearate, plant or animal stearic acid, lauric acid, dimethylpolysiloxane (also dimethicone), methylpolysiloxane (methicone), simethicone (a mixture of dimethylpolysiloxane with an average chain length of 200 to 350 dimethylsiloxane units and silica gel) or alginic acid. These organic surface coatings can occur alone, in combination and/or in combination with inorganic coating materials.

Furthermore, it can be advantageous, according to the invention, to use pearlescent pigments.

These include natural pearlescent pigments such as
“Fish silver” (mixed guanine/hypoxanthine crystals from fish scales) and
“Mother of pearl” (ground seashells),
   monocrystalline pearlescent pigments such as bismuthoxychloride (BiOCl), layer-substrate pigments: e.g. mica/metallic oxide

The basis for pearlescent pigments are, for example, powdered pigments or castor oil dispersions of bismuthoxychloride and/or titanium dioxide and bismuthoxychloride and/or titanium dioxide on mica. Especially advantageous, for instance, is the glossy pigment listed under CIN 77163.

The following types of pearlescent pigments based on mica/metallic oxide, for example, are also advantageous:

| | Overlay/layer | |
|---|---|---|
| Group | thickness | Color |
| Silver-white pearlescent | TiO₂: 40-60 nm | silver |
| pigments | | |
| Interference pigments | TiO₂: 60-80 nm | yellow |
| | TiO₂: 80-100 nm | red |
| | TiO₂: 100-140 nm | blue |
| | TiO₂: 120-160 nm | green |
| Glossy color pigments | Fe₂O₃ | bronze |
| | Fe₂O₃ | copper |
| | Fe₂O₃ | red |
| | Fe₂O₃ | red- |
| | | violet |
| | Fe₂O₃ | red- |
| | | green |
| | Fe₂O₃ | black |
| Combination pigments | TiO₂/Fe₂O₃ | gold |
| | | tones |
| | TiO₂/Cr₂O₃ | green |
| | TiO₂/Berlin blue | deep |
| | | blue |
| | TiO₂/carmine | red |

Especially preferred are the pearlescent pigments from Merck that can be obtained under the commercial name Timiron, Colorona or Dichrona.

The list of named pearlescent pigments is not to be limiting, of course. In the sense of the present invention, pearlescent pigments can be obtained in a multitude of known ways. For example, other substrates besides mica can be coated with additional metallic oxides such as silica and the like. For example, SiO₂ particles coated with TiO₂ and Fe₂O₃ (“Ronaspheres”), which are sold by Merck, are advantageous.

It can also be advantageous to dispense with a substrate such as mica entirely. Especially preferred are pearlescent pigments manufactured using SiO₂. Such pigments, which can also have goniochromatic effects, can be obtained, for example, under the commercial name Sicopearl Fantastico from BASF.

Pigments from Engelhard/Mearl based on calcium sodium borosilicate, which are coated with titanium dioxide, can also be advantageously used. These can be obtained under the name Reflecks. Because of their particle size of 40-180 μm, they also lend a glistening effect to the color.

The dyes and pigments can be present both individually and in mixture as well as layered next to each other, whereby different color effects are generally produced by different coating thicknesses.

The especially preferred pigment dyes according to the invention are the blue pigments listed, such as INCI: Cl 77007, Outremer Supercosmetique W 6803 from Les Colorants Wackherr, INCI: Cl 77891+mica+silica, Timiron Splendid Blue from Merck.

Also preferred according to the invention are mica coated with titanium dioxide and silicon dioxide, such as INCI: mica+Cl 77891+silica, Timiron Arctic Silver from Merck, INCI: mica+Cl 77891, Timiron Gleamer Flake MP-45 from Merck; aluminum oxide or silicon dioxide coated with titanium oxide and tin oxide, such as INCI: silica+Cl 77891+tin oxide, Xirona Magic Mauve from Merck; for instance INCI: alumina+Cl 77891+tin oxide, Xirona Silver, mica coated with titanium oxide and Berlin blue, for instance INCI: mica+Cl 77891+Cl 77510, Colorona Light Blue and Colorona Dark Blue from Merck.

### Pearlescent:

Also preferred is the use of pearlescent agents based on dialkyl ethers which are solid at 30° C. and have the following formula, for example: R—O—R′. R and R′ can be the same or different, straight-chained or branched, saturated or unsaturated alkyl radicals. These can consist of 12 to 30 carbon atoms, preferably of 14 to 24 carbon atoms. It is especially preferred that R and R′ consist of a stearyl radical (e.g. INCI: distearyl ether, Cutina STE from Cognis).

The dialkyl ethers used are not water soluble at a concentration of over 0.1% at 25° C.

Preferably, acylated radicals consisting of a fatty acid chain with 8 to 30 carbon atoms can also be used.

Each acylated derivative contains at least one RC(═O) group, wherein R is a fatty acid chain with 8 to 30 carbon atoms.

Especially ethylene glycol monostearates and ethylene glycol distearates, e.g. INCI: glycol distearates, Cutina AGS from Cognis, INCI: water+glycol distearates+glycerin+laureth-4+cocamidopropyl betaine, Euperlan PK 3000 OK from Cognis, INCI: PEG-3 distearates, Cutina TS from Cognis.

These are advantageously used according to the invention in a concentration between 0.5% and 2%.

### Opacifiers:

It is also advantageously possible according to the invention to use opacifiers. Preferred are the sodium salts of a polymer of styrene with a monomer consisting of acrylic acid, methacrylic acid or another olefin and one of its esters, e.g. INCI: sodium styrene/acrylate copolymer, Acusol OP 301 from Rohm & Haas.

These are advantageously used according to the invention in a concentration between 0.5% and 2%.

The multicolored cosmetic preparation according to the invention contains one or more hydrocolloids/gel formers in a concentration of 0.1 to 8 wt. %, preferably from 0.2 to 6 wt. % and especially preferably from 0.3 to 4 wt. % in relation to the total weight of the preparation.

“Hydrocolloid” is the technological abbreviated name for the more correct name “hydrophilic colloid”. Hydrocolloids, also called thickeners or gel formers, are macromolecules that have a largely linear form and have intermolecular interaction abilities that enable secondary and primary valence bonds between the individual molecules and therefore the formation of a network-like structure. They are partially water soluble, natural or synthetic polymers that form gels or viscous solutions in aqueous systems. They increase the viscosity of the water by either binding water molecules (hydration) and/or by taking the water up into their interwoven macromolecules and enveloping it, simultaneously limiting the mobility of the water. Such water soluble polymers represent a large chemical group of very different natural and synthetic polymers whose common feature is their solubility in water or aqueous media. The prerequisite for this is that these polymers possess a sufficient number of hydrophilic groups for water solubility and are not too highly networked. The hydrophilic groups can be non-ionic, anionic or cationic, for example, as follows:

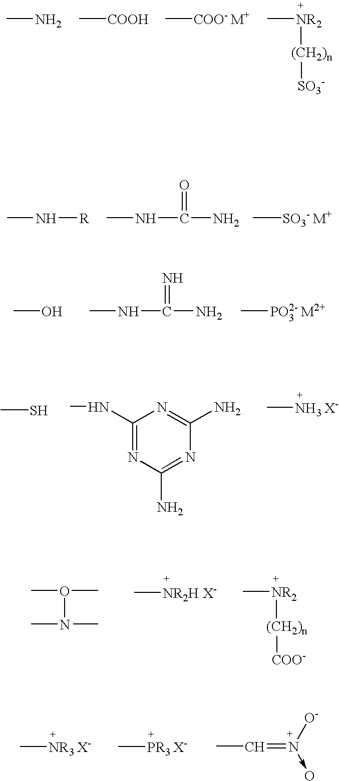

The group of cosmetic and dermatologically relevant hydrocolloids can be divided as follows:

organic, natural compounds such as agar-agar, carrageen, tragacanth, gum arabic, alginates, pectins, polyoses, guar gum, locust bean gum, starch, dextrins, gelatin, casein, organic modified natural substances such as carboxymethylcellulose and other cellulose ethers, hydroxyethyl cellulose and propylcellulose and the like, organic fully synthetic compounds such as polyacrylic and polymethacrylic compounds, vinylpolymers, polycarboxylic acids, polyethers, polyimines, polyamides, inorganic compounds such as polysilicic acids, clay minerals such as montmorillonites, zeolites, silicic acids.

Agar agar, carrageen, tragacanth, gum arabic, alginates, pectins, polyoses, guar gum, locust bean gum, starch, dextrins, gelatin, casein, cellulose ethers, derivatives of hydroxyethyl and hydroxyethylpropyl cellulose, polyacrylic and polymethacrylic compounds, vinylpolymers, polycarboxylic acids, polyethers, polyimines, polyamides, polysilicic acids, clay minerals, zeolites, and silicic acids are used as advantageous hydrocolloids according to the invention.

Preferred hydrocolloids according to the invention are, for example, methylcelluloses, which is what the methyl ethers of cellulose are called. They are characterized by the following structural formula,

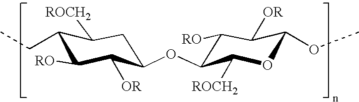

in which R can represent a hydrogen or a methyl group.

Especially advantageous in the sense of the present invention are the mixed ethers of cellulose, also generally designated as methyl celluloses, which include, along with a predominant content of methyl groups, 2-hydroxyethyl, 2-hydroxypropyl or 2-hydroxybutyl groups as well. Especially preferred are (hydroxypropyl)methyl celluloses, for example those that can be obtained under the commercial name Methocel E4M from Dow Chemical Company.

Also advantageous according to the invention is sodium carboxymethyl cellulose, the sodium salt of the glycolic acid ether of cellulose, for which R in structural formula I can represent a hydrogen and/or CH₂—COONa. Especially preferred is the sodium carboxymethyl cellulose that can be obtained under the commercial name Natrosol Plus 330 CS from Aqualon, also called cellulose gum.

Also preferred in the sense of the present invention is xanthan (CAS No. 11138-66-2), also called xanthan gum, which is an anionic heteropolysaccharide and is usually formed by the fermentation of corn sugar and isolated as a potassium salt. It is produced by Xanthomonas campestris and a few other species under aerobic conditions with a molecular weight of 2×10⁶ to 24×10⁶. Xanthan is formed from a chain with β-1,4-bound glucose (cellulose) with side chains. The structure of the subgroups consists of glucose, mannose, glucuronic acid, acetate and pyruvate. Xanthan is the name for the first microbial anionic heteropolysaccharide. It is produced by Xanthomonas campestris and a few other species under aerobic conditions with a molecular weight of 2−15×10⁶. Xanthan is formed from a chain with β-1,4-bound glucose (cellulose) with side chains. The structure of the subgroups consists of glucose, mannose, glucuronic acid, acetate and pyruvate. The number of pyruvate units determines the viscosity of the xanthan. Xanthan is produced in two-day batch cultures with a yield of 70-90% in relation to the carbohydrate used. This produces yields of 25-30 g/l. Processing follows after killing off the culture by precipitation with 2-propanol, for example. Xanthan is then dried and ground.

Another advantageous gel former in the sense of the present invention is carrageen, a gel former and extract synthesized from North Atlantic red algae (Chondrus crispus and Gigartina stellata) in a manner similar to agar.

Often, the name carrageen is used for the dried algae product and carrageenan for the extract from this product. The carrageen precipitated from the hot water extract of the algae is a colorless to sand-colored powder with a molecular weight ranging from 100,000-800,000 and a sulfate content of about 25%. Carrageen, which is very soluble in warm water, forms a thixotropic gel when cooled, even if the water content is 95-98%. The solidity of the gel is caused by the double helix structure of the carrageen. Carrageenan is divided into three main components: The gel building K-fraction consists of D-galactose-4-sulfate and 3,6-anhydro-α-D-galactose, which are glycosidically bound alternating in 1,3- and 1,4-position (on the other hand, agar contains 3,6-anhydro-α-L-galactose). The non-gelling λ-fraction is composed of 1,3-glycosidically linked D-galactose-2-sulfate and 1,4-bound D-galactose-2,6-disulfate groups and is readily soluble in cold water. The t-carrageenan formed from D-galactose-4-sulfate in a 1,3-bond and 3,6-anhydro-α-D-galactose-2-sulfate in a 1,4-bond is both water soluble and gel building. Other carrageen types are designated with Greek letters: α, β, γ, μ, ν, ξ, π, ω, χ. The type of cations present (K⁺, NH₄⁺, Na⁺, Mg²⁺, Ca²⁺) also influences the solubility of the carrageens.

Polyacrylates are also advantageous for use as gelators in the sense of the present invention. Advantageous polyacrylates according to the invention are acrylate/alkylacrylate copolymers, especially those selected from the group of so-called carbomers or carbopols (Carbopol® is actually a registered trademark of NOVEON, Inc.). The advantageous acrylate/alkylacrylate copolymer(s) according to the invention are characterized especially by the following structure:

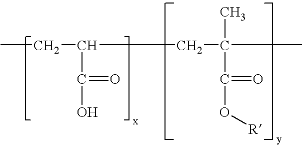

In this structure, R′ represents a long-chain alkyl group and x and y are numbers that symbolize the respective stoichiometric portion of the respective comonomers.

According to the invention, acrylate copolymers and/or acrylate/alkylacrylate copolymers which can be obtained under the commercial names Carbopol® 1382, Carbopol® 981 and Carbopol® 5984, Aqua SF-1 from NOVEON, Inc. or Aculyn® 33 from International Specialty Products Corp. are preferred.

Also advantageous are copolymers from C10-C30 alkylacrylates and one or more monomers of acrylic acid, methacrylic acids or their esters, which are crosslinked with an allyl ether of saccharose or an allyl ether of pentaerythritol

Compounds bearing the INCI designation “acrylates/C10-30 alkyl acrylates crosspolymers” are advantageous. Especially advantageous are those that can be obtained under the commercial name Pemulen TR1 and Pemulen TR2 from NOVEON, Inc.

Other advantageous compounds are those bearing the INCI designation “acrylates/C12-24 pareth-25 acrylate copolymer” (can be obtained under the commercial name Synthalen® W2000 from 3V, Inc.), those bearing the INCI designation “acrylates/steareth-20 methacrylate copolymer” (can be obtained under the commercial name Aculyn® 22 from International Specialty Products Corp.), those bearing the INCI designation “acrylates/steareth-20 itaconate copolymer” (can be obtained under the commercial name Structure 2001® from National Starch), those bearing the INCI designation “acrylates/aminoacrylates/C10-30 alkyl PEG-20 itaconate copolymer” (can be obtained under the commercial name Structure Plus® from National Starch) and similar polymers.

The especially preferred hydrocolloids according to the invention are: Acrylates copoly-mer (AQUA SF-1), acrylates/C10-30 alkyl acrylates crosspolymer (Carbopol ETD 2020), xanthan gum (Kelter).

According to the invention, the ratio of the total quantity of pigment dyes to the total quantity of hydrocolloids advantageously is from 1:100 to 1:4, preferably from 1:200 to 6:1 and especially preferably from 1:300 to 1:8.

It is advantageous according to the invention if the water content of the preparation is 20 to 95 wt. %, preferably 30 to 90 wt. % and most preferably 40 to 85 wt. %, in relation to the total weight of the preparation.

The preparations according to the invention may advantageously contain one or more surfactants. Anionic, cationic, non-ionic and zwitterionic surfactants can all be used advantageously according to the invention.

Advantageous detersive anionic surfactants in the sense of the present invention are acylamino acids and their salts such as
acylglutamates, especially sodium acylglutamate
sarcosinates, for example myristoyl sarcosin, TEA-lauroyl sarcosinate, sodium lauroyl sarcosinate and sodium cocoyl sarcosinate,

Sulfonic acids and their salts, such as
acylisethionates, e.g. sodium/ammonium cocoyl isethionate,
sulfosuccinates, for example dioctyl sodium sulfosuccinate, disodium laureth sulfosuccinate, disodium lauryl sulfosuccinate and disodium undecylenamido MEA-sulfosuccinate, disodium PEG-5 laurylcitrate sulfosuccinate and derivatives,
   as well as sulfuric acid esters such as
alkyl ether sulfate, for example, sodium-, ammonium-, magnesium-, MIPA- and TIPA laureth sulfate, sodium myreth sulfate and sodium C₁₂₋₁₃ pareth sulfate,
alkyl sulfates, for example, sodium-, ammonium- and TEA lauryl sulfate.

Advantageous detersive cationic surfactants in the sense of the present invention are quaternary surfactants. Quaternary surfactants contain at least one N atom covalently bound to 4 alkyl or aryl groups. Advantageous, for example, are alkylbetaine, alkylamidopropylbetaine and alkylamidopropylhydroxysultaine.

Advantageous detersive amphoteric surfactants in the sense of the present invention are
acyl/dialkylethylenediamines, for example sodium acylamphoacetate, disodium acylamphodipropionate, disodium alkylamphodiacetate, sodium acylamphohydroxypropylsulfonate, disodium acylamphodiacetate and sodium acylamphopropionate,

Advantageous detersive non-ionic surfactants in the sense of the present invention are
alkanolamides such as cocamides MEA/DEA/MIPA,
esters formed by the esterification of carboxylic acids with ethylene oxide, glycerin, sorbitan or other alcohols,
ethers, for example ethoxylated alcohols, ethoxylated lanolin, ethoxylated polysiloxanes, propoxylated POE ether and alkylpolyglycosides such as laurylglucoside, decylglycoside and cocoglycoside.

Other advantageous anionic surfactants are
taurates, for example sodium lauroyl taurate and sodium methylcocoyl taurate,
ether carboxylic acids, for example sodium laureth-13 carboxylate and sodium PEG-6 cocamide carboxylate, sodium PEG-7 olive oil carboxylate
phosphoric acid esters and salts such as DEA oleth-10 phosphate and dilaureth-4 phosphate,
alkyl sulfonates, for example sodium coconut monoglyceride sulfate, sodium C₁₂₋₁₄ olefinsulfonate, sodium laurylsulfoacetate and magnesium PEG-3 cocamide sulfate.

Other advantageous amphoteric surfactants are
N-alkylamino acids, for example aminopropylalkylglutamide, alkylaminopropionic acid, sodium alkylimidodipropionate and lauroamphocarboxyglycinate and N-coconut fatty acid amidoethyl-N-hydroxyethylglycinate sodium salts and their derivatives.

Additional advantageous non-ionic surfactants are alcohols.

Additional suitable anionic surfactants in the sense of the present invention are also
acylglutamates such as di-TEA-palmitoyl aspartate and sodium caprylic/capric glutamate,
acylpeptides, for example palmitoyl hydrolyzed milk protein, sodium cocoyl hydrolyzed soy protein and sodium/potassium cocoyl hydrolyzed collagen
   as well as carboxylic acids and derivatives such as
a lauric acid, aluminum stearate, magnesium alkanolate and zinc undecylenate,
ester carboxylic acids, for example calcium stearoyl lactylate, laureth-6 citrate and sodium PEG-4 lauramide carboxylate,
alkylarylsulfonate.

Additional suitable cationic surfactants in the sense of the present invention are also
alkylamines,
alkylimidazoles,
ethoxylated amines,
   especially their salts.

Additional suitable non-ionic surfactants in the sense of the present invention are also amine oxides such as cocoamidopropylamine oxide.

It is advantageous to select the detersive surfactant(s) according to the invention from the group of surfactants having an HLB value greater than 25; especially advantageous are those that have an HLB value greater than 35.

Preferred according to the invention are alkyl sulfates or alkyl ether sulfates or surfactant combinations of alkyl ether sulfates with amphoteric or non-ionic surfactants, wherein a surfactant combination of alkyl ether sulfates with alkylamidopropylbetaines or alkylamphoacetates or alkylpolyglucosides is especially preferred. Especially preferred are also combinations of alkyl ether sulfates with alkylamidopropylbetaines or alkylamphoacetates and acyl glutamates.

It is most especially preferred, according to the invention, to use alkyl ether sulfates or a combination of alkyl ether sulfate and alkylamidopropylbetaine or alkyl ether sulfate and alkyl polyglucoside as surfactants.

It is advantageous in the sense of the present invention if the content of one or more detersive surfactants in the cosmetic preparation ranges from 1 to 30 wt. %, preferably from 5 to 25 wt. %, most especially advantageously from 10 to 20 wt. %, in relation to the total weight of the preparation.

According to the invention, the preparations according to the invention can advantageously contain polysorbates. Polysorbates represent a class of compounds derived from sorbitan, a furan derivative obtained from sorbitol by splitting off two molecular equivalents of water. The hydroxyl groups of sorbitan are etherified with polyethylene glycols whose ends may be esterified with fatty acids. They can be generally represented by the formula

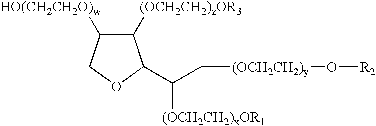

R₁, R₂, R₃=H, fatty acid radical.

Advantageous polysorbates in the sense of the invention are, for example, the
polyoxyethylene(20)sorbitanmonolaurate (Tween 20, CAS No. 9005-64-5)
polyoxyethylene(4)sorbitanmonolaurate (Tween 21, CAS No. 9005-64-5)
polyoxyethylene(4)sorbitanmonostearate (Tween 61, CAS No. 9005-67-8)
polyoxyethylene(20)sorbitantristearate (Tween 65, CAS No. 9005-71-4)
polyoxyethylene(20)sorbitanmonooleate (Tween 80, CAS No. 9005-65-6)
polyoxyethylene(5)sorbitanmonooleate (Tween 81, CAS No. 9005-65-5)
polyoxyethylene(20)sorbitantrioleate (Tween 85, CAS No. 9005-70-3).

These are advantageously used according to the invention individually or as a mixture of several polysorbates in a concentration of 0.1 to 5 wt. % and especially in a concentration of 1.5 to 2.5 wt. % relative to the total weight of the preparation.

The cosmetic preparation may contain several oil phases along with one or more water phases and exist, for example, in the form of W/O, O/W, W/O/W or O/W/O emulsions. Such formulations can preferably also be a microemulsion (e.g. a PIT emulsion), a solid emulsion (i.e. an emulsion stabilized by solids, e.g. a Pickering emulsion), wherein transparent or translucent microemulsions are particularly preferred according to the invention.

The preparation according to the invention can also contain, according to the invention, other ingredients as an aqueous solution or aqueous phase of an emulsion along with water, for example alcohols, diols or polyols with a low carbon atom count, as well as their ethers, preferably ethanol, isopropanol, propylene glycol, glycerin, ethylene glycol, ethylene glycol monoethyl ether or ethylene glycol monobutyl ether, propylene glycol monomethyl ether, propylene glycol monoethyl ether or propylene glycol monobutyl ether, diethylene glycol monomethyl ether or diethylene glycol monoethyl ether and analogous products, also alcohols with a low carbon atom count, e.g. ethanol, isopropanol, 1,2-propandiol and glycerin.

The preparations according to the invention can advantageously contain moistening or moisture retaining agents (so-called moisturizers). Advantageous moisture retaining agents in the sense of the present invention are, for example, glycerin, lactic acid and/or lactates, especially sodium lactate, butylene glycol, propylene glycol, biosaccharide gum-1, soy glycine, ethylhexyloxyglycerin, pyrrolidone carboxylic acid and urea. It is also especially an advantage to use polymer moisturizers from the group of polysaccharides that are water soluble and/or can be swelled in water and/or gelled using water. Especially advantageous are, for example, hyaluronic acid, chitosan and/or a fucose-rich polysaccharide, filed in Chemical Abstracts under register number 178463-23-5 and which can be obtained, for example, as Fucogel®1000 from SOLABIA S.A.

The cosmetic or dermatological preparations according to the invention can also advantageously contain fillers, although this is not obligatory. These fillers further improve the sensory and cosmetic properties of the formulations, for example, and evoke or intensify a velvety or silky skin feeling. Advantageous fillers in the sense of the present invention are starch and starch derivatives (such as tapioca starch, distarch phosphate, aluminum or sodium starch octenyl succinate and similar substances), pigments that have neither a primary UV filtering nor a coloring effect (such as boronitride, etc.) and/or Aerosils® (CAS No. 7631-86-9).

An additional content of antioxidants is generally preferred. According to the invention, all antioxidants suitable or customary for cosmetic and/or dermatological use can be used as advantageous antioxidants.

Water soluble antioxidants can be specially advantageously used in the sense of the present invention, for example vitamins, e.g. ascorbic acid and its derivatives.

Preferred antioxidants also include vitamin E and its derivatives as well as vitamin A and its derivatives.

The quantity of antioxidants (one or more compounds) in the preparations is preferably 0.001 to 30 wt. %, more preferably 0.05 to 20 wt. %, especially 0.1 to 10 wt. %, in relation to the total weight of the preparation.

If vitamin E and/or its derivatives are the antioxidant(s), it is advantageous to select the respective concentrations in the range from 0.001 to 10 wt. % in relation to the total weight of the formulation.

If the antioxidant(s) are vitamin A or vitamin A derivatives or carotenes or their derivatives, it is advantageous to select their respective concentrations from the range of 0.001 to 10 wt. % in relation to the total weight of the formulation.

It is especially advantageous if the cosmetic preparations according to the present invention contain cosmetic or dermatological agents wherein preferred agents are antioxidants that can protect the skin from oxidative stress.

It is advantageous to select the antioxidants from the group consisting of amino acids (e.g. glycine, lysine, arginine, cystein, histidine, tyrosine, tryptophan) and their derivatives (as salt, ester, ether, sugar, nucleotide, nucleoside, peptide and lipid compounds), imidazoles (e.g. urocanic acid) and its derivatives (as salt, ester, ether, sugar, nucleotide, nucleoside, peptide and/or lipid compounds), peptides such as D,L-carnosine, D-carnosine, L-carnosine, anserine and its derivatives (e.g. as salt, ester, ether, sugar, thiole, nucleotide, nucleoside, peptide and lipid compounds), carotinoids, carotenes (e.g. α-carotene, β-carotene, ψ-lycopene, phytoene) and their derivatives (e.g. as salt, ester, ether, sugar, nucleotide, nucleoside, peptide and/or lipid compounds), chlorogenic acid and its derivatives (as salt, ester, ether, sugar, thiole, nucleotide, nucleoside, peptide and/or lipid compounds), aurothioglucose, propylthiouracil and other thioles (e.g. thioredoxin, lipoic acid, glutathione, cysteine, cystine, cystamine and its glycosyl, N-acetyl, methyl, ethyl, propyl, amyl, butyl and lauryl, palmitoyl, oleyl, γ-linoleyl, cholesteryl and glyceryl esters) and their salts, dilaurylthiodipropionate, distearylthiodipropionate, thiodipropionic acid and its derivatives (as salt, ester, ether, sugar, thiole, nucleotide, nucleoside, peptide and/or lipid compounds) as well as sulfoximine compounds (e.g. homocysteine sulfoximine, buthionine sulfone, penta-, hexa-, heptathionine sulfoximine) in very small tolerable doses (e.g. pmol to μmol/kg). Also (metal) chelators (e.g. apoferritin, desferral, lactoferrin, α-hydroxy fatty acids, palmitic acid, phytic acid) and their derivatives (as salt, ester, ether, sugar, thiole, nucleotide, nucleoside, peptide and/or lipid compounds), α-hydroxy acids (e.g. citric acid, lactic acid, malic acid), humic acid, bile acid, bile extracts, bilirubin, biliverdin, EDTA, EGTA and its derivatives, unsaturated fatty acids and their derivatives (e.g. γ-linolenic acid, linoleic acid, oleic acid), folic acid and its derivatives, furfurylidene sorbitol and its derivatives, ubichinone, ubichinol, plastochinone and its derivatives (as salt, ester, ether, sugar, thiole, nucleotide, nucleoside, peptide and lipid compounds), vitamin C and derivates (e.g. ascorbyl palmitate, Mg-ascorbyl phosphate, ascorbyl acetate), tocopherols and derivatives (e.g. vitamin E acetate), vitamin A and its derivates, vitamin D and its derivatives, vitamin E and its derivatives, vitamin F and its derivatives as well as phenolic compounds and plant extracts, these containing, for example, flavonoids (e.g. glycosyl rutin, ferulic acid, caffeic acid), furfurylidene glucitol, butylhydroxytoluol, butylhydroxyanisol, nordihydroguaiacum resin acid, nordihydro guajaret acid, trihydroxybutyrophenone and its derivatives (as salt, ester, ether, sugar, nucleotide, nucleoside, peptide and lipid compounds). Uric acid and its derivatives, mannose and its derivatives (as salt, ester, ether, sugar, thiole, nucleotide, nucleoside, peptide and lipid compounds). Zinc and its derivatives (e.g. ZnO, ZnSO₄), selenium and its derivatives (e.g. selenium methionine, ebselen), stilbenes and their derivatives (e.g. stilbene oxide, transstilbene oxide) and the suitable derivatives according to the invention of these substances (as salt, ester, ether, sugar, thiole, nucleotide, nucleoside, peptide and/or lipid compounds).

Additional advantageous agents in the sense of the present invention are natural substances and/or their derivatives, such as alpha-lipoic acid, phytoene, D-biotin, coenzyme Q10, alpha-glucosyl rutin, carnitine, carnosin, natural and/or synthetic isoflavonoids, creatine, taurine, and/or β-alanine.

Formulations according to the invention containing, for example, known anti-wrinkle agents such as flavone glycosides (especially α-glycosyl rutin), coenzyme Q10, vitamin E and/or derivates and similar compounds are especially advantageously suited for the prophylaxis and treatment of cosmetic or dermatological skin changes such as those that occur in the aging of the skin (such as dryness, roughness and the formation of wrinkles from dryness, itching, reduced lubrication (e.g. after washing), visible vascular expansion (teleangiectasia, cuperosis), flaccidness and the formation of lines and wrinkles, local hyper-, hypo- and malpigmentation (e.g. age spots), increased sensitivity to mechanical stress (e.g. chapping) and similar changes). These are also advantageously suitable against the appearance of dry or rough skin.

In the preparations according to the invention, however, other pharmaceutically or dermatologically active substances such as substances that calm and nourish the skin can also be incorporated. These include, for example, panthenol, allantoin, tannin, antihistamines, antiphlogistics, glucocorticoids (e.g. hydrocortisone) and plant substances such as azulene and bisabolol, glycyrrhizine, hamamelin and plant extracts such as chamomile, aloe vera, hamamelis and licorice root.

The quantity of previously named agents, antioxidants, etc. (one or more compounds) in the preparations is preferably 0.001 to 30 wt. %, more preferably 0.05-20 wt. %, and especially 1-10 wt. % in relation to the total weight of the preparation.

The oil phase of the formulations according to the invention is advantageously selected from the groups of polar oils, for example from the group of lecithins and the fatty acid triglycerides, namely the triglycerine esters of saturated and/or unsaturated, branched and/or unbranched alkane carboxylic acids of a chain length of 8 to 24, especially 12 to 18 carbon atoms. The fatty acid triglycerides can be advantageously selected, for instance, from the group of synthetic, half-synthetic and natural oils such as coconut glyceride, olive oil, sunflower oil, soy oil, peanut oil, rapeseed oil, almond oil, palm oil, coconut oil, castor oil, wheat germ oil, grapeseed oil, safflower oil, evening primrose oil, macadamia nut oil and many similar substances.

Also advantageous according to the invention are, for example, natural waxes of animal and plant origin such as bee's wax and other insect waxes as well as berry wax, shea butter and/or lanolin.

Additional advantageous polar oil components can also be selected in the sense of the present invention from the group of the esters of saturated and/or unsaturated, branched and/or unbranched alkane carboxylic acids of a chain length of 3 to 30 carbon atoms and saturated and/or unsaturated, branched and/or unbranched alcohols of a chain length of 3 to 30 carbon atoms as well as from the group of the esters of aromatic carboxylic acids and saturated and/or unsaturated, branched and/or branched alcohols of a chain length of 3 to 30 carbon atoms. Such ester oils can then be advantageously selected from the group including octylpalmitate, octylcocoate, octylisostearate, octyldodeceylmyristate, octyldodecanol, cetearylisononanoate, isopropylmyristate, isopropylpalmitate, isopropyl-stearate, isopropyloleate, n-butylstearate, n-hexyllaurate, n-decyloleate, isooctylstearate, isononylstearate, isononylisononanoate, 2-ethylhexylpalmitate, 2-ethylhexyllaurate, 2-hexyldecylstearate, 2-octyidodecylpalmitate, stearylheptanoate, oleyloleate, oleylerucate, erucyloleate, erucylerucate, tridecylstearate, tridecyltrimellitate, and synthetic, half-synthetic and natural mixtures of such esters, such as jojoba oil.

The oil phase can also be advantageously selected from the group of dialkylethers and dialkylcarbonates; for example, dicaprylylether (Cetiol OE) and/or dicaprylylcarbonate, which can be obtained under the commercial name Cetiol CC from Cognis, are advantageous.

It is also preferred that the oil components from the group including isoeicosane, neopentyl glycol diheptanoate, propylene glycol dicaprylate/dicaprate, caprylic/capric/diglycerylsuccinate, butylene glycol dicaprylate/dicaprate, cocoglycerides (e.g. Myritol® 331 from Henkel), C₁₂₋₁₃-alkyllactate, di-C₁₂₋₁₃-alkyltartrate, triisostearin, dipentaerythrityl hexacaprylate/hexacaprate, propylene glycol monoisostearate, tricaprylin, dimethylisosorbid. It is especially advantageous if the oil phase of the formulations according to the invention features a content of C₁₂₋₁₅ alkylbenzoate or consists totally of this.

Advantageous oil components are also, for example, butyloctylsalicylate (for example, the one that can be obtained under the commercial name Hallbrite BHB from CP Hall), hexadecylbenzoate and butyloctylbenzoate and mixtures of these (Hallstar AB) and/or diethylhexylnaphthalate (Corapan®TQ from Haarmann & Reimer).

Any blends of such oil and wax components are also advantageous to use in the sense of the present invention.

The oil phase can also advantageously contain non-polar oils, for example those selected from the group of branched and unbranched carbohydrates and waxes, especially mineral oil, vaseline (petrolatum), paraffin oil, squalane and sqaulene, polyolefins, hydrogenated polyisobutenes and isohexadecane. Among the polyolefins, polydecenes are the preferred substances.

The oil phase can also advantageously feature a content of cyclic or linear silicone oils or consist totally of such oils, whereby it is preferred, however, that an additional content of other oil phase components besides the silicone oil or silicone oils be used.

Silicone oils are high molecular weight synthetic polymeric compounds in which silicon atoms are linked by oxygen atom chains and/or network-like with each other, and the remaining valences of the silicon are saturated by carbohydrate groups (mostly methyl, rarely ethyl, propyl, phenyl groups, among others). Silicone oils are designated systematically as polyorganosiloxanes. The methyl-substituted polyorganosiloxanes, which represent the most significant compounds of this group with regard to quantity and are characterized by the following structural formula

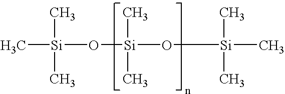

are also designated as polydimethylsiloxane or dimethicone (INCI). There are dimethicones in different chain lengths or with different molecular weights.

Especially advantageous polyorganosiloxanes in the sense of the present invention are, for example, dimethylpolysiloxanes [poly(dimethylsiloxane)], which can be obtained, for example, under the commercial names Abil 10 through 10,000 from Th. Goldschmidt. Also advantageous are phenylmethylpolysiloxanes (INCI: phenyl dimethicone, phenyl tri-methicone), cyclic silicones (octamethylcyclotetrasiloxane and decamethylcyclopenta-siloxane), which are also designated as cyclomethicones according to the INCI, amino-modified silicones (INCI: amodimethicones) and silicon waxes, e.g. polysiloxane/poly-alkylene copolymers (INCI: stearyl dimethicones and cetyl dimethicones) and dialkoxydi-methylpolysiloxanes (stearoxy dimethicones and behenoxy stearyl dimethicones), which can be obtained as different Abil wax types from Th. Goldschmidt. Other silicon oils are also advantageous to use in the sense of the present invention, for example cetyl dimethicone, hexamethylcyclotrisiloxane, polydimethylsiloxane, poly(methylphenylsiloxane).

The preparations according to the invention can also contain all water soluble and/or oil soluble UVA, UVB and/or broad band filter substances approved according to the cosmetic ordinance.

Accordingly, the preparations in the sense of the present invention preferably contain at least one UVA and/or UVB filter substance. The formulations can also contain, if needed, one or more organic and/or inorganic pigments as UV filter substances, which can be present in the water and/or oil phase, although this is not obligatory.

Preferred inorganic pigments are metal oxides and/or other metal compounds which are scarcely soluble or insoluble in water, especially oxides of titanium (TiO₂), zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals as well as blends of such oxides, and the sulfate of barium (BaSO₄).

Titanium dioxide pigments can be present in both the crystal modifications rutile and anatase and can be advantageously surface-treated (coated) in the sense of the present invention, thus forming or maintaining, for example, a hydrophilic, amphiphilic or hydrophobic character. This surface treatment can consist of the pigments being provided with a thin inorganic and/or organic hydrophilic and/or hydrophobic layer according to processes already known. The different surface coatings can also contain water in the sense of the present invention.

The described coated and uncoated titanium dioxides can, in the sense of the present invention, also be used in the form of commercially obtainable oily or aqueous predispersions. These predispersions can be advantageously added as dispersion aids and/or solubilization facilitators.

The titanium dioxides according to the invention are characterized by a primary particle size between 10 nm and 150 nm.

| Commercial | | Additional ingredient | |
|---|---|---|---|
| Name | Coating | for predispersion | Manufacturer |
| MT-100TV | Aluminum hydroxide | — | Tayca Corporation |
| | Stearic acid | | |
| MT-100Z | Aluminum hydroxide | — | Tayca Corporation |
| | Stearic acid | | |
| MT-100F | Stearic acid | — | Tayca Corporation |
| | Iron oxide | | |
| MT-500SAS | Alumina, silica | — | Tayca Corporation |
| | Silicon | | |
| MT-100AQ | Silica | — | Tayca Corporation |
| | Aluminum hydroxide | | |
| | Alginic acid | | |
| Eusolex T-2000 | Alumina | — | Merck KgaA |
| | Simethicone | | |
| Eosolex TS | Alumina, stearic acid | — | Merck KgaA |
| Titanium dioxide | None | — | Degussa |
| P25 | | | |
| Titanium dioxide | Octyltrimethylsilane | — | Degussa |
| T805 | | | |
| (Uvinul TiO₂) | | | |
| UV-Titanium | Alumina | — | Kemira |
| X170 | Dimethicone | | |
| UV-Titanium | Alumina, silica | — | Kemira |
| X161 | Stearic acid | | |
| Tioveil AQ 10PG | Alumina | Water | Solaveil |
| | Silica | Propylene glycol | Uniquema |
| Mirasun TiW 60 | Alumina | Water | Rhone-Poulenc |
| | Silica | | |

In the sense of the present invention, especially preferred titanium dioxides are MT-100 Z and MT-100 TV from Tayca Corporation, Eusolex T-2000 and Eusolex TS from Merck and the titanium dioxide T 805 from Degussa.

Zinc oxides can also be used in the sense of the present invention in the form of commercially obtainable oily or aqueous predispersions. Suitable zinc oxide particles and predispersion of zinc oxide particles according to the invention are characterized by a primary particle size of <300 nm and can be obtained under the following commercial names from the companies listed:

| Commercial Name | Coating | Manufacturer |
|---|---|---|
| Z-Cote HP1 | 2% dimethicone | BASF |
| Z-Cote | / | BASF |
| ZnO NDM | 5% dimethicone | H&R |
| MZ 707M | 7% dimethicone | M. Tayca Corp. |
| Nanox 500 | / | Elementis |
| ZnO Neutral | / | H&R |

Especially preferred zinc oxides in the sense of the invention are the Z-Cote HP1 from BASF and the zinc oxide NDM from Haarmann & Reimer.

The total quantity of one or more inorganic pigments in the finished cosmetic preparation is advantageously selected in the range from 0.1 wt. % to 25 wt. %, preferably from 0.5 wt. % to 18 wt. %.

An advantageous organic pigment in the sense of the present invention is 2,2′-methylene-bis-(6-(2H-benzotriazol-2-yl)-4-(1,1,3,3-tetramethylbutyl)-phenol) [INCI: bisoctyltriazol], which can be obtained under the commercial name Tinosorb® M from CIBA-Chemikalien GmbH.

Advantageous UVA filter substances in the sense of the present invention are dibenzoyl-methane derivates, especially 4-(tert.-butyl)-4′-methoxydibenzoylmethane (CAS No. 70356-09-1), which is sold by Givaudan under the commercial name Parsol® 1789 and by Merck under the commercial name Eusolex® 9020.

Additional advantageous UVA filter substances are phenylene-1,4-bis-(2-benzimidazyl)-3,3′-5,5′-tetrasulfonic acid and its salts, especially the corresponding sodium, potassium or triethanolammonium slats, especially phenylene-1,4-bis-(2-benzimidazyl)-3,3′-5,5′-tetrasulfonic acid-bis-sodium salt with the INCI designation bisimidazylate, which can be obtained, for example, under the commercial name Neo Heliopan AP from Haarmann & Reimer.

Also advantageous are 1,4-di(2-oxo-10-sulfo-3-bornylidenmethyl)benzene and its salts (especially the corresponding 10-sulfato compounds, especially the corresponding sodium, potassium or triethanol ammonium salt), which is also designated as benzene-di(2-oxo-3-bornylidenmethyl-10-sulfonic acid).

Additional advantageous UVA filter substances are hydroxybenzophenones, characterized by the following structural formula:

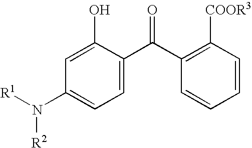

wherein
R¹ and R² represent hydrogen, C₁-C₂₀-alkyl, C₃-C₁₀-cycloalkyl or C₃-C₁₀-cycloalkenyl, whereby the substituents R¹ and R² can form a 5-ring or 6-ring together with the nitrogen atom to which they are bonded
R³ represents a C₁-C₂₀-alkyl group.

An especially advantageous hydroxybenzophenone in the sense of the present invention is hexyl 2-(4′-diethylamino-2′-hydroxybenzoyl)-benzoate (also: aminobenzophenone), which can be obtained under the commercial name Uvinul A Plus from BASF.

Advantageous UV filter substances in the sense of the present invention are also the so-called broadband filters, i.e. filter substances that absorb both UVA and UVB rays.

Advantageous broadband filters or UVB filter substances are, for example, bis-resorcinyltriazine derivatives with the following structure:

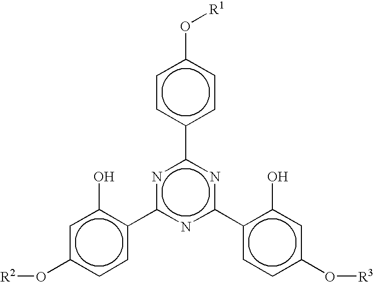

wherein R¹, R² and R³ represent and are selected independently of each other from the group of branched and unbranched alkyl groups with 1 to 10 carbon atoms or an individual hydrogen atom. Especially preferred are 2,4-bis-{[4-(2-ethyl-hexyloxy)-2-hydro-xy]-phenyl}-6-(4-methoxyphenyl)-1,3,5-triazine (INCI: aniso triazine), which can be obtained under the commercial name Tinosorb® S from CIBA-Chemikalien GmbH.

Especially advantageous preparations in the sense of the present invention that feature a high to very high UVA protection preferably contain several UVA and/or broadband filters, especially dibenzoylmethane derivatives [for example, 4-(tert.-butyl)-4′-methoxydiben-zoylmethane], benzotriazole derivates [for example 2, 2′-methylene-bis-(6-(2H-benzotriazol-2-yl)-4-(1,1,3,3-tetramethylbutyl)-phenol)], phenylene-1,4-bis-(2-benzimi-dazyl)-3,3′-5,5′-tetrasulfonic acid and or its salts, 1,4-di(2-oxo-10-sulfo-3-bornyliden-methyl)-benzene and/or its salts and/or 2,4-bis-{[4-(2-ethyl-hexyloxy)-2-hydroxyl-phenyl}-6-(4-methoxyphenyl)-1,3,5-triazine, each individually or in any combinations with each other.

Other UV filter substances as well, which feature the structural motif

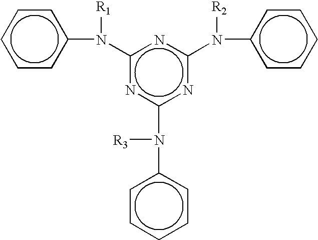

are advantageous UV filter substances in the sense of the present invention, for example those s-triazine derivatives described in the European Patent Application EP 570 838 A1 whose chemical structure is represented by the general formula

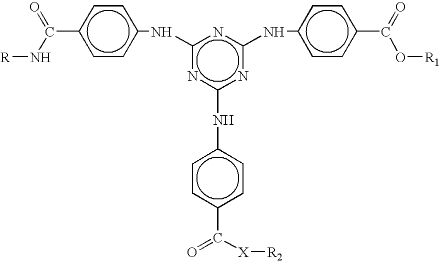

wherein
R represents a branched or unbranched C₁-C₁₈-alkyl group, a C₅-C₁₂ cycloalkyl group, optionally substituted with one or more C₁-C₄-alkyl groups,
X represents an oxygen atom or an NH group,
R₁ represents a branched or unbranched C₁-C₁₈-alkyl group, a C₅-C₁₂-cycloalkyl group, substituted, if necessary, with one or more C₁-C₄— alkyl groups, or a hydrogen atom, an alkali metal atom, an ammonium group or a group of the formula

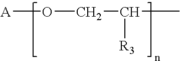

in which
A represents a branched or unbranched C₁-C₁₈-alkyl group, a C₅-C₁₂ cycloalkyl or aryl group, substituted, optionally, with one or more C₁-C₄— alkyl groups,
R₃ represents a hydrogen atom or a methyl group,
n represents a number from 1 to 10,
R₂ represents a branched or unbranched C₁-C₁₈-alkyl group, a C₅-C₁₂-cycloalkyl group, substituted, optionally, with one or more C₁-C₄— alkyl groups, if X represents the NH group, and
a branched or unbranched C₁-C₁₈-alkyl group, a C₅-C₁₂-cycloalkyl group, substituted, optionally, with one or more C₁-C₄— alkyl groups, or a hydrogen atom, an alkali metal atom, an ammonium group or a group of the formula

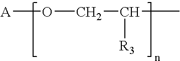

in which
A represents a branched or unbranched C₁-C₁₈-alkyl group, a C₅-C₁₂ cycloalkyl or aryl group, substituted, optionally, with one or more C₁-C₄— alkyl groups,
R₃ represents a hydrogen atom or a methyl group,
n represents a number from 1 to 10,
if X represents an oxygen atom.

Especially preferred UV filter substances in the sense of the present invention are also an asymmetrically substituted s-triazine, whose chemical structure is represented by the formula

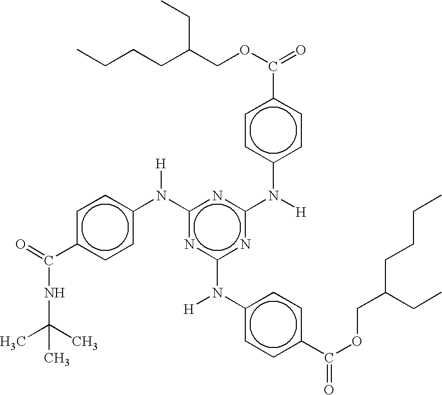

which is also called dioctylbutylamidotriazone (INCI: dioctylbutamidotriazone) in the following, and can be obtained under the commercial name UVASORB HEB from Sigma 3V.

Also advantageous in the sense of the present invention is a symmetrically substituted s-triazine, tris(2-ethylhexyl) 4,4′,4″-(1,3,5-triazine-2,4,6-triyltriimino)-tris-benzoate, synonym: 2,4,6-tris-[anilino-(p-carbo-2′-ethyl-1′-hexyloxy)]-1,3,5-triazine (INCI: octyl triazone), which is sold by BASF under the trademark UVINUL® T 150.

In the European Patent Application 775 698 as well, preferably used bis-resorcinyltriazine derivatives are described whose chemical structure is represented by the generic formula

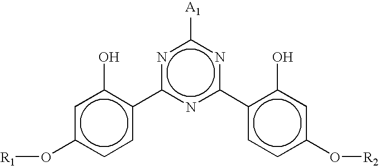

whereby R₁, R₂ and A₁ represents the most different organic groups.

Also advantageous in the sense of the present invention are the sodium salt of 2,4-bis-{[4-(3-sulfonato)-2-hydroxy-propyloxy)-2-hydroxy]-phenyl}-6-(4-methoxyphenyl)-1,3,5-triazine, 2,4-bis-{[4-(3-(2-propyloxy)-2-hydroxy-propyloxy)-2-hydroxy]-phenyl}-6-(4-meth-oxyphenyl)-1,3,5-triazine, 2,4-bis-{[4-(2-ethyl-hexyloxy)-2-hydroxy]-phenyl}-6-[4-(2-meth-oxyethyl-carboxyl)-phenylamino]-1,3,5-triazine, 2,4-bis-{[4-(3-(2-propyloxy)-2-hydroxy-propyloxy)-2-hydroxy]-phenyl}-6-[4-(2-ethyl-carboxyl)-phenylamino]-1,3,5-triazine, 2,4-bis-{[4-(2-ethyl-hexyloxy)-2-hydroxy]-phenyl}-6-(1-methyl-pyrrol-2-yl)-1,3,5-triazine, 2,4-bis-{[4-tris(trimethylsiloxy-silylpropyloxy)-2-hydroxy]-phenyl}-6-(4-methoxyphenyl)-1,3,5-triazin, 2,4-bis-{[4-(2″-methylpropenyloxy)-2-hydroxy]-phenyl}-6-(4-methoxyphenyl)-1,3,5-triazine and 2,4-bis-{[4-(1′,1′,1′,3′,5′,5′,5′-heptamethylsiloxy-2″-methyl-propyloxy)-2-hydroxy]-phenyl}-6-(4-methoxyphenyl)-1,3,5-triazine.

An advantageous broadband filter in the sense of the present invention is 2,2′-methylene-bis-(6-(2H-benzotriazol-2-yl)-4-(1,1,3,3-tetramethylbutyl)-phenol), which can be obtained under the commercial name Tinosorb® M from CIBA-Chemikalien GmbH.

Another advantageous broadband filter in the sense of the present invention is 2-(2H-benzotriazol-2-yl)-4-methyl-6-[2-methyl-3-[1,3,3,3-tetramethyl-1-[(trimethylsilyl)oxy]di-siloxanyl]propyl]-phenol (CAS No.: 155633-54-8) with the INCI designation drometrizole trisiloxane.

The UVB and/or broadband filters can be oil soluble or water soluble. Advantageous oil soluble UVB and/or broadband filter substances are, for example:
3-benzylidene camphor derivatives, preferably 3-(4-methylbenzylidene)camphor, 3-benzylidene camphor;
4-aminobenzoic acid derivatives, preferably (2-ethylhexyl) 4-(dimethylamino)-benzoate, amyl 4-(dimethylamino)benzoate;
2,4,6-trianilino-(p-carbo-2′-ethyl-1′-hexyloxy)-1,3,5-triazine;
ester of benzalmalonic acid, preferably di(2-ethylhexyl) 4-methoxybenzalmalate;
esters of cinnamic acid, preferably (2-ethylhexy) 4-methoxycinnamate, isopentyl 4-methoxycinnamate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone
and UV filters bound to polymers.

Advantageous water soluble UVB and/or broadband filter substances are, for example:
Salts of 2-phenylbenzimidazol-5-sulfonic acid such as its sodium, potassium, or triethanolammonium salt, as well as the sulfonic acid salt itself;
Sulfonic acid derivatives of the 3-benzylidene camphors, such as 4-(2-oxo-3-bornyli-denemethyl)benzenesulfonic acid, 2-methyl-5-(2-oxo-3-bornylidenemethyl)sulfonic acid and its salts.

Especially advantageous UV filter substance in the sense of the present invention which are liquid at room temperature are homomethylsalicylate (INCI: homosalate), 2-ethyl-hexyl-2-hydroxybenzoate (2-ethylhexylsalicylate, octylsalicylate, INCI: octyl salicylate), 4-isopropylbenzylsalicylate and esters of cinnamic acid, preferably 4-methoxy cinnamic acid (2-ethylhexyl)ester (2-ethylhexyl-4-methoxycinnamate, INCI: octyl methoxycinnamate) and 4-methoxy cinnamic acid isopentylester (isopentyl-4-methoxycinnamate, INCI: isoamyl p-methoxycinnamate), 3-(4-(2,2-bis ethoxycarbonylvinyl)-phenoxy)propenyl)-methoxysiloxane/dimethylsiloxane-copolymer (INCI: dimethicodiethylbenzalmalonate) which can be obtained, for example, under the commercial name Parsol® SLX from Hoffmann La Roche.

Another light protection filtering substance for advantageous use according to the invention is ethylhexyl-2-cyano-3,3-diphenylacrylate (octocrylene), which can be obtained from BASF under the name Uvinul® N 539.

It can also be a great advantage to use polymer-bound or polymeric UV filter substances in preparations according to the present invention, especially those described in WO-A-92/20690.

The list of named UV filters that can be used in the sense of the present invention is not intended to be exhaustive, of course.

It is an advantage for the preparations according to the invention to contain substances that absorb UV rays in the UVA and/or UVB range in a total quantity of, for example, 0.1 wt. % to 30 wt. %, preferably 0.5 to 20 wt. %, especially 1.0 to 15.0 wt. %, in relation to the total weight of the preparations, in order to provide cosmetic preparations that protect the hair or skin from the entire range of ultraviolet radiation. They can also serve as sun protectors for the hair or skin.

The compositions contain, according to the invention, in addition to the previously named substances, if necessary, the customary adjuvants for cosmetics, for example, perfume, dyes, antimicrobial substances, lubricating agents, complexing and sequestering agents, pearlescing agents, additional plant extracts, vitamins, minerals, anti-scaling agents, active agents, preservatives, bactericides, repellants, self-tanners, depigmentation agents, pigments that have a coloring effect, softening, moistening and/or moisture-retaining substances or other customary ingredients of a cosmetic or dermatological formulation such as emulsifiers, polymers, foam stabilizers and electrolytes.

The preparation according to the invention can advantageously contain, according to the invention, one or more preservatives. Advantageous preservatives in the sense of the present invention are, for example, formaldehyde splitters (such as DMDM hydantoin, which can be obtained, for example, under the commercial name Glydant™ from Lonza), iodopropylbutylcarbamate (which can be obtained, for example, under the commercial name Glycacil-L, Glycacil-S from Lonza and/or Dekaben LMB from Jan Dekker), parabens (i.e. p-hydroxybenzoic acid alkylesters such as methyl-, ethyl-, propyl- and/or butylparaben), phenoxyethanol, ethanol, benzoic acid and other similar substances. Normally, the preservative system according to the invention includes additional advantageous preservatives such as octoxyglycerin, soy glycine, etc. The following table gives an overview of some advantageous preservatives according to the invention:

| | |
|---|---|
| E 200 | Sorbic acid |
| E 201 | Sodium sorbate |
| E 202 | Potassium sorbate |
| E 203 | Calcium sorbate |
| E 210 | Benzoic acid |
| E 211 | Sodium benzoate |
| E 212 | Potassium benzoate |
| E 213 | Calcium benzoate |
| E 214 | p-Hydroxybenzoic acid ethylester |
| E 215 | p-Hydroxybenzoic acid ethylester sodium |
| | salt |
| E 216 | p-Hydroxybenzoic acid-n-propylester |
| E 217 | p-Hydroxybenzoic acid-n-propylester sodium |
| | salt |
| E 218 | p-Hydroxybenzoic acid methylester |
| E 219 | p-Hydroxybenzoic acid methylester sodium |
| | salt |
| E 220 | Sulfur dioxide |
| E 221 | Sodium sulfite |
| E 222 | Sodium hydrogen sulfite |
| E 223 | Sodium disulfite |
| E 224 | Potassium disulfite |
| E 226 | Calcium sulfite |
| E 227 | Calcium hydrogen sulfite |
| E 228 | Potassium hydrogen sulfite |
| E 230 | Biphenyl (diphenyl) |
| E 231 | Orthophenylphenol |
| E 232 | Sodium |
| | orthophenylphenolate |
| E 233 | Thiabendazol |
| E 235 | Natamycin |
| E 236 | Formic acid |
| E 237 | Sodium formiate |
| E 238 | Calcium formiate |
| E 239 | hexamethylentetramine |
| E 249 | Potassium nitrite |
| E 250 | Sodium nitrite |
| E 251 | Sodium nitrate |
| E 252 | Potassium nitrate |
| E 280 | Propionic acid |
| E 281 | Sodium propionate |
| E 282 | Calcium propionate |
| E 283 | Potassium propionate |
| E 290 | Carbon dioxide |

Also advantageous are preservatives or preservative additives customary for cosmetics such as dibromdicyanobutane (2-bromo-2-brommethylglutarodinitrile), phenoxyethanol, 3-iodo-2-propynylbutylcarbamate, 2-bromo-2-nitro-propane-1,3-diol, imidazolidinyl urea, 5-chloro-2-methyl-4-isothiazolin-3-one, 2-chloroacetamide, benzalkonium chloride, benzyl alcohol.

It is especially preferred, according to the invention, if benzoic acid and/or salicylic acid and/or their derivatives or salts are used as preservatives.

Parabens and mixtures with phenoxethanol/DMDM hydantoin are preferred according to the invention.

Advantageous according to the invention are one or more preservatives contained in the preparation according to the invention in a concentration of 2 wt. % or less than 2 wt. %, preferably 1.5 wt. % or less than 1.5 wt. % and more preferably 1 wt. % or less than 1 wt. %, each in relation to the total weight of the preparation in the composition featured at the time of the application to the substrate.

The preparations according to the invention advantageously contain one or more conditioners. Conditioners which are preferred according to the invention are, for example, all those compounds listed in the International Cosmetic Ingredient Dictionary and Handbook (Volume 4, Publisher: R. C. Pepe, J. A. Wenninger, G. N. McEwen, The Cosmetic, Toiletry, and Fragrance Association, 9th Edition, 2002) under Section 4 under the keywords Hair Conditioning Agents, Humectants, Skin-Conditioning Agents, Skin-Conditioning Agents-Emollient, Skin-Conditioning Agents-Humectant, Skin-Conditioning Agents-Miscellaneous, Skin-Conditioning Agents-Occlusive and Skin Protectants, as well as all those compounds listed in EP 0934956 (pp. 11-13) under water soluble conditioning agent and oil soluble conditioning agent. A part of these compounds are listed by name among the components of the aqueous and oil phases. Additional advantageous conditioners according to the invention are represented, for example, by the compounds called polyquaternium according to the international nomenclature for cosmetic ingredients (INCI) (especially polyquaternium-1 through polyquaternium-56). Conditioners which are preferred according to the invention can be taken, for example, from the following list:

| Designation according | | | Example (commercial |
|---|---|---|---|
| to the INCI | | Polymer type | name) |
| | CAS Number | | |
| Polyquaternium-2 | CAS 63451-27-4 | Urea, N,N′-bis[3- | Mirapol ® A-15 |
| | | (dimethylamino)propyl]-, | |
| | | polymer with 1,1′- | |
| | | oxybis(2-chloroethane) | |
| Polyquaternium-5 | CAS 26006-22-4 | Acrylamide, β- | |
| | | methacryloxyethyltriethyl | |
| | | ammoniummethosulfate | |
| | CAS-Nummer | | |
| Polyquaternium-6 | CAS 26062-79-3 | N,N-dimethyl-N-2- | Merquat ® 100 |
| | | propenyl-2-propene- | |
| | | aminiumchloride | |
| Polyquaternium-7 | CAS 26590-05-6 | N,N-dimethyl-N-2- | Merquat ® S |
| | | propenyl-2-propene- | |
| | | aminiumchloride, 2- | |
| | | propenamide | |
| Polyquaternium-10 | CAS 53568-66-4, | Quaternary ammonium | Celquat ® SC-230M |
| | 55353-19-0, 54351-50- | salt of hydroxyethyl | |
| | 7, 68610-92-4, 81859- | cellulose | |
| | 24-7 | | |
| Polyquaternium-11 | CAS 53633-54-8 | Vinylpyrrolidone/dimethyl | Gafquat ® 755N |
| | | aminoethyl- | |
| | | methacrylate-copolymer/ | |
| | | diethylsulfate reaction | |
| | | product | |
| Polyquaternium-16 | CAS 29297-55-0 | Vinylpyrrolidone/ | Luviquat ® HM552 |
| | | vinylimidazolinummetho | |
| | | chloride copolymer | |
| Polyquaternium-17 | CAS 90624-75-2 | | Mirapol ® AD-1 |
| Polyquaternium-19 | CAS 110736-85-1 | Quaternized, water | |
| | | soluble polyvinyl alcohol | |
| Polyquaternium-20 | CAS 110736-86-2 | Quaternized | |
| | | polyvinyloctadecylether | |
| | | dispersible in water | |
| Polyquaternium-21 | | Polysiloxane- | Abil ® B 9905 |
| | | polydimethyl- | |
| | | dimethylammonium | |
| | | acetate-copolymer | |
| Polyquaternium-22 | CAS 53694-17-0 | Dimethyldiallylammoniumchloride/ | Merquat ® 280 |
| | | acrylic acid | |
| | | copolymer | |
| Polyquaternium-24 | CAS 107987-23-5 | Polymeric, quaternary | Quartisoft ® LM-200 |
| | | ammonium salt of the | |
| | | hydroxyethylcellulose | |
| Polyquaternium-28 | CAS 131954-48-8 | Vinylpyrrolidone/ | Gafquat ® HS-100 |
| | | methacrylamidopropyltri | |
| | | methylammoniumchloride- | |
| | | copolymer | |
| Polyquaternium-29 | CAS 92091-36-6, | Chitosan, with propylene | Lexquat ® CH |
| | 148880-30-2 | oxide added and | |
| | | quaternized with | |
| | | epichlorohydrine | |
| Polyquaternium-31 | CAS 136505-02-7, | Polymeric, quaternary | Hypan ® QT 100 |
| | 139767-67-7 | ammonium salt, | |
| | | manufactured by the | |
| | | conversion of DMAPA- | |
| | | acrylate/acrylic | |
| | | acid/acrylonitrogen | |
| | | copolymers and | |
| | | diethylsulfate | |
| Polyquaternium-32 | CAS 35429-19-7 | N,N,N-trimethyl-2-{[82- | |
| | | methyl-1-oxo-2- | |
| | | propenyl)oxy]- | |
| | | ethanaminiumchloride, | |
| | | polymer with 2- | |
| | | propenamide | |
| Polyquaternium-37 | CAS 26161-33-1 | | |
| Polyquaternium-44 | | Copolymeric, quaternary | |
| | | ammonium salt of | |
| | | vinylpyrrolidone and | |
| | | quaternized imidazolin | |

Additional advantageous film formers according to the invention are represented by cellulose derivatives and quaternized guar gum derivatives, especially guar hydroxypropylammoniumchloride (e.g. Jaguar Excel®, Jaguar C 162® from Rhodia, CAS 65497-29-2, CAS 39421-75-5).

Non-ionic poly-N-vinylpyrrolidone/polyvinylacetate copolymers (e.g. Luviskol VA 64W®, BASF), anionic acrylate copolymers (e.g. Luviflex Soft®, BASF), and/or amphoteric amide/acrylate/methacrylate copolymers (e.g. Amphomer®, National Starch) can also be used advantageously according to the invention.

It is also an advantage to add complex formers to the preparations according to the invention. Complex formers are advantageously selected from the group consisting of ethylenediamine tetraacetic acid (EDTA) and its anions, nitrilotriacetic acid (NTA) and its anions, hydroxyethylene diaminotriacetic acid (HOEDTA) and its anions, diethylene aminopentaacetic acid (DPTA) and its anions, trans-1,2-diaminocyclohexantetraacetic acid (CDTA) and its anions, tetrasodium iminodisuccinate, trisodium ethylenediamine disuccinate.

It is an advantage according to the invention to add vitamins, plant extracts and UV light protection filters to the preparation according to the invention. Thus, the addition of calcium vitamin complexes, for example, is especially advantageous according to the invention.

It is especially advantageous according to the invention if the multicolored cosmetic preparation according to the invention is made of one or more individual color layers or color zones that are homogenous within themselves and that border on each other horizontally and/or vertically or are partially mixed in a marbled or spiral pattern. The homogenous color layers or color zones are advantageously formed according to the invention from portions of the preparation according to the invention.

It is therefore preferable, according to the invention, if the partial stirring of the multicolored preparations according to the invention leads to a marbled or striated appearance of the products, whereby the color areas are visibly separated from one another.

The preparation according to the invention can advantageously contain, according to the invention, additional dyes and/or effect materials, for example color particles, gas bubbles, active ingredient capsules, glitter materials and other larger objects (generally called effect materials).

It is also advantageous according to the invention if at least one formulation is present in the form of an emulsion.

It is in keeping with the invention to store the cosmetic preparation according to the invention in a transparent, translucent and/or dyed package and to use it from this package.

It is advantageous according to the invention if the cosmetic preparation according to the invention is stored in a bottle, squeeze bottle, pump dispenser, pump spray bottle or aerosol bottle and is used from this bottle. Accordingly, bottles, squeeze bottles, pump dispensers, pump spray bottles or aerosol bottles containing a preparation according to the invention are also according to the invention.

The use of the preparation according to the invention as a cleaning preparation for the skin and/or skin appendages, as a hair conditioner or styling gel is according to the invention.

The use of the cosmetic preparation according to the invention as a foaming cleansing preparation is according to the invention.

Especially the use of the cosmetic preparation according to the invention as a shampoo, shower gel, washing lotion and/or bath cleanser is according to the invention.

The preparation according to the invention can also be used advantageously as a skin care product or decorative cosmetic.

In addition, the preparation according to the invention is outstandingly well suited for the cleaning of everyday objects (e.g. dishes, table and cupboard surfaces, autos, clothing, laundry).

The following examples are intended to explain the compositions according to the invention without intending to limit the invention to these examples. The numerical values in the examples represent weight percents in relation to the total weight of the respective preparations.

Here, example formulations from formulation part A—shower emulsion—can be combined with each other in any manner in order to achieve the described effects. The same applies for the formulations from formulation part B—shower gel—: The portions of the formulations do not have to be the same.

Formulations from Part A—shower emulsion—can also be combined with those from Part B—shower gel—.

One part can be colorless and one part dyed or white. Clear and emulsion-like parts can also be combined.

Portions from formulation part C—styling gel—can also be combined in any manner with formulations from Part D—styling gels.

Formulation Part A - Shower Emulsion -

| | 1 | 2 | 3 | 4 | 5 |
|---|---|---|---|---|---|
| Sodium laurylether sulfate | 10 | 9 | 8 | — | 10 |
| Sodium myristyleth sulfate | | | | 8 | |
| Alkylamidopropylbetaine | — | 3 | 5 | 2 | — |
| Sodium acylglutamate | — | — | 2 | — | — |
| Alkylpolyglucoside | — | — | — | 2 | 1.5 |
| Acrylate copolymer (acrylates/C10-30 alkyl | 0.65 | 0.5 | 0.3 | 0.6 | 0.7 |
| acrylate crosspolymer) | | | | | |
| Phenoxethanol + methylparaben + | 0.8 | 0.8 | 0.8 | 0.8 | 0.8 |
| butylparaben + ethylparaben + | | | | | |
| isobutylparaben + propylparaben | | | | | |
| Soy oil | 6 | 40 | 25 | — | 30 |
| Paraffin oil | 35 | 5 | 18 | 45 | 13 |
| Almond oil | 2 | — | — | — | — |
| Quaternary ammonium salt of hydroxyethyl | — | — | 0.10 | — | — |
| cellulose | | | | | |
| Ethoxylated glycerin fatty acid ester (PEG-7 | — | 0.5 | — | 1 | 1 |
| glyceryl cocoate) | | | | | |
| CI 77007 | — | 0.3 | — | — | 1.5 |
| CI 77891 + mica + silica | — | — | — | 0.075 | — |
| Water + CI 42051 | — | — | 0.3 | — | — |
| Unispheric (lactose + cellulose + | 0.2 | — | — | — | — |
| hydroxypropyl methylcellulose + CI 77007) | | | | | |
| NaOH | q.s. | q.s. | q.s. | q.s. | q.s. |
| Perfume | 1.0 | 1.2 | 1.0 | 1.0 | 0.8 |
| Water | ad 100 | ad 100 | ad 100 | ad 100 | ad 100 |

Formulation Part B - Shower Gel -

| | 1 | 2 | 3 | 4 | 5 | 6 | 7 | 8 | 9 |
|---|---|---|---|---|---|---|---|---|---|
| Sodium laurylether sulfate | 13.0 | 11.0 | 9.0 | 8.5 | 12.0 | 10 | 11 | — | 10 |
| Alkylamidopropylbetaine | 0.50 | 1.5 | 2.0 | 1.0 | | 4.0 | 2.5 | 4.0 | 4.0 |
| Alkylpolyglucoside | — | — | — | — | 1.10 | — | — | 4.0 | — |
| Sodium cocoylglutamate | 1.50 | 0.5 | 1.0 | 0.5 | 0.75 | 2.0 | 3.0 | 1.5 | 2.0 |
| Acrylate copolymer | 3.00 | 1.50 | 1.75 | 2.00 | 2.20 | 2.40 | 3.5 | 2.8 | 2.4 |
| (acrylates copolymer) | | | | | | | | | |
| Quaternary ammonium | — | — | — | 0.20 | — | — | — | — | — |
| salt of hydroxyethyl | | | | | | | | | |
| cellulose | | | | | | | | | |
| PEG-6 caprylic/capric | — | 0.75 | 1.0 | — | — | 1.0 | — | — | — |
| glycerides | | | | | | | | | |
| PEG-40 hydrated castor | 1.0 | 1.0 | 1.0 | 1.0 | 1.0 | 1.0 | 1.0 | 1.2 | — |
| oil | | | | | | | | | |
| Glycol distearate | 1.0 | — | — | — | — | — | — | — | — |
| Styrene/acrylate | — | 0.5 | — | — | — | — | — | — | 0.5 |
| copolymer | | | | | | | | | |
| DMDM hydantoin | 0.30 | 0.30 | 0.30 | 0.30 | | 0.30 | 0.30 | 0.30 | |
| Methylparaben | — | — | — | — | 0.40 | — | — | — | 0.40 |
| Propylparaben | — | — | — | — | 0.20 | — | — | — | 0.20 |
| Phenoxyethanol | — | — | — | — | 0.60 | — | — | — | 0.60 |
| Unispheric (lactose + cellulose + | 0.3 | — | — | — | — | 0.20 | — | — | — |
| hydroxypropyl | | | | | | | | | |
| methylcellulose + CI 77007) | | | | | | | | | |
| Titanium dioxide | — | — | — | — | — | — | — | — | 0.3 |
| Timiron Artic Silver | — | — | — | — | — | — | 0.05 | — | — |
| Mica + CI 77891 | — | — | — | 0.05 | — | — | — | — | — |
| Mica + CI 77891 + CI | — | — | 0.025 | — | — | — | — | — | — |
| 77510 | | | | | | | | | |
| Polyethylene | — | — | — | — | — | — | — | — | 0.2 |
| Water + CI 42051 | — | 0.05 | — | — | — | — | — | — | — |
| Citric acid | q.s. | q.s. | q.s. | q.s. | q.s. | q.s. | q.s. | q.s. | q.s. |
| NaOH | q.s. | q.s. | q.s. | q.s. | q.s. | q.s. | q.s. | q.s. | q.s. |
| Perfume | 1.0 | 0.5 | 1.2 | 1.0 | 0.8 | 1.0 | 0.8 | 1.0 | 1.0 |
| Water | ad | ad | ad | ad | ad | ad | ad | ad | ad |
| | 100 | 100 | 100 | 100 | 100 | 100 | 100 | 100 | 100 |

Formulation Part C - Styling Gel -

| | 1 | 2 | 3 | 4 |
|---|---|---|---|---|
| Carbomer | 0.85 | 1.0 | 0.85 | 1.0 |
| CI 77007 | — | 0.3 | — | — |
| CI 77891 + mica + silica | — | — | — | 0.075 |
| Water + CI 42051 | — | — | 0.3 | — |
| Unispheric (lactose + cellulose + | 0.2 | | | |
| hydroxypropyl methylcellulose + | | | | |
| CI 77007) | | | | |
| VP/VA copolymers | 6.0 | 8.0 | 6.0 | 8.0 |
| Dimethicone | 0.2 | 0.3 | 0.2 | 0.3 |
| Sodium benzoate | — | — | 0.5 | 0.5 |
| Ethanol | 10.0 | 10.0 | — | — |
| PEG-40 hydrated castor oil | — | — | 0.2 | 0.2 |
| NaOH | q.s. | q.s. | q.s. | q.s. |
| Perfume | q.s. | q.s. | q.s. | q.s. |
| Water | ad 100 | ad 100 | ad 100 | ad 100 |

Formulation Part D - Styling Gel -

| | 1 | 2 | 3 | 4 |
|---|---|---|---|---|
| Carbomer | 0.85 | 1.0 | 0.85 | 1.0 |
| Titanium dioxide | — | 0.3 | — | — |
| Timiron Artic Silver | — | — | — | 0.075 |
| Mica + CI 77891 | — | — | 0.3 | — |
| Mica + CI 77891 + CI 77510 | 0.2 | | | |
| VP/VA copolymers | 6.0 | 8.0 | 6.0 | 8.0 |
| Dimethicone | 0.2 | 0.3 | 0.2 | 0.3 |
| Sodium benzoate | — | — | 0.5 | 0.5 |
| Ethanol | 10.0 | 10.0 | — | — |
| PEG-40 hydrated castor oil | — | — | 0.2 | 0.2 |
| NaOH | q.s. | q.s. | q.s. | q.s. |
| Perfume | q.s. | q.s. | q.s. | q.s. |
| Water | ad 100 | ad 100 | ad 100 | ad 100 |

1.-11. (canceled)

12. A multicolored cosmetic preparation, wherein the preparation comprises
(a) one or more pigment dyes,
(b) one or more hydrocolloids,
(c) water,
and wherein the preparation has a yield point of from 0.5 Pa to 40 Pa and a tan δ of from 0.02 to 0.8.

13. The preparation of claim 12, wherein the preparation comprises (a) in an amount of from 0.001 to 2 wt. %, relative to a total weight of the preparation.

14. The preparation of claim 12, wherein the preparation comprises (a) in an amount of from 0.001 to 1 wt. %, relative to a total weight of the preparation.

15. The preparation of claim 12, wherein the preparation comprises (b) in an amount of from 0.1 to 8 wt. %, relative to a total weight of the preparation.

16. The preparation of claim 12, wherein the preparation comprises (b) in an amount of from 0.2 to 6 wt. %, relative to a total weight of the preparation.

17. The preparation of claim 12, wherein the preparation has a yield point of from 0.5 Pa to 20 Pa.

18. The preparation of claim 12, wherein the preparation has a tan δ of from 0.05 to 0.6.

19. The preparation of claim 12, wherein component (b) comprises one or more of acrylates copolymer (AQUA SF-1), acrylates/C10-30 alkyl acrylate cross-polymer (Carbopol ETD 2020) and xanthan gum (Kelter).

20. The preparation of claim 12, wherein the preparation further comprises one or more surfactants selected from anionic, cationic, non-ionic and amphoteric surfactants.

21. The preparation of claim 20, wherein the preparation comprises the one or more surfactants in an amount of from 1 to 30 wt. %, relative to a total weight of the preparation.

22. The preparation of claim 20, wherein the one or more surfactants comprise at least one of (i) one or more alkylsulfates (ii) one or more alkylether sulfates and (iii) a surfactant combination of one or more alkylether sulfates with one or more amphoteric or non-ionic surfactants.

23. The preparation of claim 20, wherein the one or more surfactants comprise at least one of (i) one or more alkylether sulfates, (ii) a combination of one or more alkylether sulfates and one or more alkylamidopropylbetains and (iii) a combination of one or more alkylether sulfates and one or more alkylpolyglucosides

24. The preparation of claim 12, wherein the preparation is in a form which comprises one or more individual color layers or color zones which are homogenous within themselves and border at least one of horizontally, vertically and spiral-like on each other, or are present partially mixed in a marbled form, with the colored areas being visibly separated from each other.

25. The preparation of claim 12 in combination with a packaging from which it can be dispensed and which is at least one of transparent, translucent and colored.

26. A cleansing preparation for skin and/or skin appendages which comprises the preparation of claim 12.

27. A multicolored cosmetic preparation, wherein the preparation comprises
(a) from 0.001 to 1 wt. %, relative to a total weight of the preparation, of one or more pigment dyes,
(b) from 0.2 to 6 wt. %, relative to a total weight of the preparation, of one or more hydrocolloids,
(c) from 20 to 95 wt. %, relative to a total weight of the preparation, of water, and wherein the preparation has a yield point of from 0.5 Pa to 20 Pa and a tan δ of from 0.05 to 0.6.

28. The preparation of claim 27, wherein the preparation comprises from 0.001 to 0.5 wt. % of (a) and from 0.3 to 4 wt. % of (b).

29. The preparation of claim 28, wherein the preparation comprises from 40 to 85 wt. % of (c).

30. The preparation of claim 28, wherein the preparation has a yield point of from 1 Pa to 6 Pa and a tan δ of from 0.1 to 0.5.

31. The preparation of claim 27, wherein component (b) comprises one or more of acrylates copolymer (AQUA SF-1), acrylates/C10-30 alkyl acrylate cross-polymer (Carbopol ETD 2020) and xanthan gum (Kelter).

32. The preparation of claim 28, wherein the preparation further comprises one or more surfactants selected from anionic, cationic, non-ionic and amphoteric surfactants in an amount of from 1 to 30 wt. %, relative to the total weight of the preparation.

33. The preparation of claim 32, wherein the one or more surfactants are present in an amount of from 5 to 25 wt. %.

34. The preparation of claim 33, wherein the one or more surfactants comprise at least one of (i) one or more alkylsulfates (ii) one or more alkylether sulfates and (iii) a surfactant combination of one or more alkylether sulfates with one or more amphoteric or non-ionic surfactants.

35. The preparation of claim 33, wherein the one or more surfactants comprise at least one of (i) one or more alkylether sulfates, (ii) a combination of one or more alkylether sulfates and one or more alkylamidopropylbetains and (iii) a combination of one or more alkylether sulfates and one or more alkylpolyglucosides.
